(19) United States
(12) Patent Application Publication
(10) Pub. No.: US 2005/0131430 A1
(43) Pub. Date: Jun. 16, 2005

(54) Suturing device

(75) Inventor: Sundaram Ravikumar, Briarcliff Manor, NY (US)

Correspondence Address: GORDON & JACOBSON, P.C., 60 LONG RIDGE ROAD, SUITE 407, STAMFORD, CT, 06902, US

(73) Assignee: Arvik Enterprises, LLC

(21) Appl. No.: 10/734,393

(22) Filed: Dec. 12, 2003

(51) Int. Cl. A61B 17/04

(52) U.S. Cl. 606/144

(57) ABSTRACT

A suturing device for surgical applications includes first and second elements that are rotatable with respect to one another about a central axis. Each element has a cutout into its exterior surface. The cutouts extend along a direction substantially parallel to the central axis and are sized to accept suture thread material therein. A central member is disposed along the central axis between the first and second elements to define space therebetween. When the first element is rotated with respect to the second element, the suture thread material wraps around the central member in the space between the first and second elements to thereby grasp and hold suture thread material therein. Preferably, the second element is realized from deformable material such that its cutout collapses and grasps suture material thread disposed therein. In this manner, the two elements cooperate to efficiently and effectively grasp and hold suture thread material therein for a broad range of suturing applications, and facilitate tension control on the suture material thread.

### BACKGROUND OF THE INVENTION

1. Field of the Invention

This invention relates broadly to suturing devices for surgical applications. More particularly, this invention relates to devices that clamp and anchor suture material to tissue.

2. State of the Art

In surgical applications it frequently necessary to anchor tissue with suture material. Typically, the suture material is coupled to a needle and sewn into the tissue surrounding a surgical site (e.g., wound). The two ends of the suture material are tensioned and knotted to provide closure of the surgical site. The ability to control the tension on the suture material is important. To this end it is common for surgeons to tie double knots, that is a first knot to control tension and a second knot to secure the first knot. Such knot tying significantly extends the time required for suturing.

There have been attempts to provide devices that take the place of conventional suturing with a suture needle and a length of suture material. Examples of devices that pinch or clamp the suture material are described in U.S. Pat. No. 2,075,508 to Davidson; U.S. Pat. No. 3,664,345 to Dabbs et al.; U.S. Pat. No. 3,976,079 to Samuels et al.; U.S. Pat. No. 4,291,698 to Fuchs et al.; U.S. Pat. No. 5,643,295 to Yoon; U.S. Pat. No. 5,720,747 to Burke; U.S. Pat. No. 5,810,853 to Yoon; U.S. Pat. No. 6,010,525 to Bonutti et al.; and U.S. Pat. No. 6,569,187 to Bonutti et al. These clamp-type devices are susceptible to inadvertent slippage of the suture material and loss of tension therein, and also have the disadvantage of requiring complex instruments, of being difficult to manipulate and/or not sufficiently reducing the time required for suturing and tying.

Thus, there remains a need in the art for devices that facilitate more time efficient and effective suturing and tying.

### SUMMARY OF THE INVENTION

It is therefore an object of the invention to provide devices and methods for suturing tissue in a time efficient and effective manner.

It is another object of the invention to provide devices and methods for suturing tissue that facilitate control over the tension applied to the suture material.

It is a further object of the invention to provide suturing devices (and corresponding methods) that are easy to manipulate.

In accord with these objects, which will be discussed in detail below, a suturing device for surgical applications includes first and second elements that are rotatable with respect to one another about a central axis. Each element has a cutout into its exterior surface. The cutouts, which extend along a direction substantially parallel to the central axis, are adapted to accept suture thread material therein. A central member is disposed along the central axis between the first and second elements to define space therebetween. When the first element is rotated with respect to the second element, the suture thread material is wrapped around the central member in the space between the first and second elements to thereby grasp and hold suture thread material. Preferably, the second element is realized with deformable material such that its cutout collapses and grasps suture material thread disposed therein.

It will be appreciated that the two elements cooperate to efficiently and effectively grasp and hold suture thread material therein for a broad range of suturing applications, and facilitate tension control on the suture material thread.

In the preferred embodiment of the invention, the device is made of bioabsorbable material for internal suturing procedures.

Additional objects and advantages of the invention will become apparent to those skilled in the art upon reference to the detailed description taken in conjunction with the provided figures.

### BRIEF DESCRIPTION OF THE DRAWINGS

FIG. 1A is an exploded view of a suturing device in accordance with the present invention;

FIG. 1B is a bottom view of the bottom element of the suturing device of FIG. 1A; and

FIG. 2 is a schematic view of an alternate suturing device in accordance with the present invention.

### DETAILED DESCRIPTION OF THE PREFERRED EMBODIMENTS

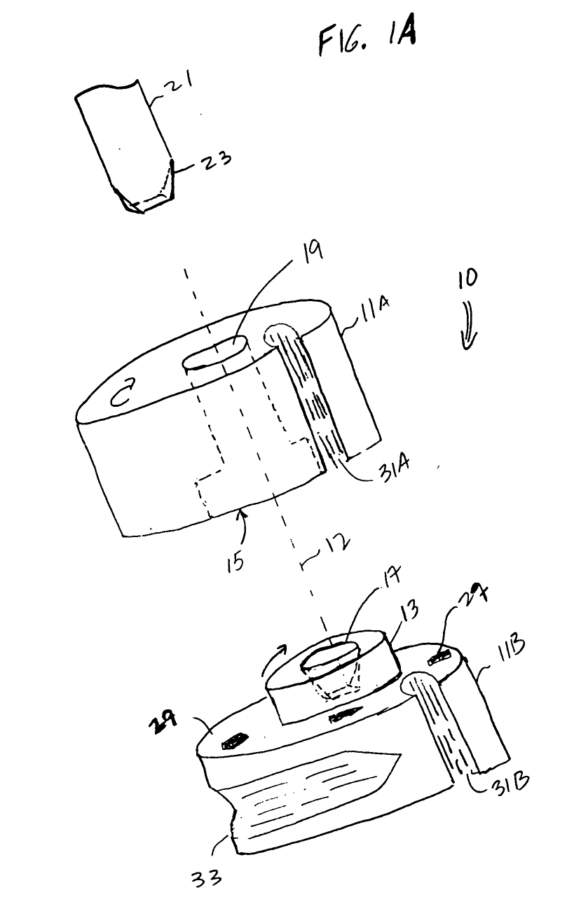

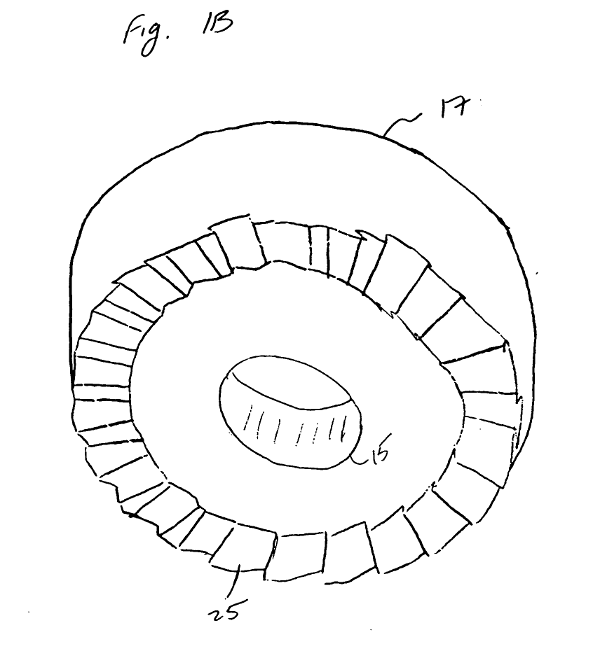

Turning now to FIGS. 1A and 1B, a suturing device in accordance with the present invention includes two elements 11A, 11B, preferably of annular shape as shown, that are rotatable with respect to one another about a central axis 12. Such rotation is preferably accomplished by affixing a rotating member 13 to the element 11B. The rotating member 13 rotates freely with respect to element 11B about the central axis 12, and snuggly fits into a bottom cavity 15 of element 11A. The rotating member 13 includes a recess 17 that is accessible through a port 19 extending along the central axis of the element 11A. The recess 17 is shaped to accept a drive tip 23 formed at the end of a mandrel 21. The mandrel 21 is inserted thru the port 19 of element 11A such that the surfaces of the drive tip 23 mate to the surfaces of the recess 17 of the rotating member 13. Rotation of mandrel 21 causes the rotating member 13 (in addition to the element 11A affixed thereto) to rotate with respect to element 11B about the central axis 12. In this manner, the two elements 11A, 11B are rotated with respect to one another.

Preferably, such rotation is limited to one direction (e.g., either counter-clockwise or clockwise) by an annular ridged surface 25 disposed on the bottom side of the rotating member 13 as shown in FIG. 1B. One or more pawls 27 are disposed on the top side 29 of the element 11B. The ridged surface 25 and the pawls 27 cooperate such that the rotation of the two elements 11A, 11B with respect to one another is limited to one direction (e.g., either counter-clockwise or clockwise).

The two elements 11A, 11B also have cutouts 31A, 31B into their exterior as shown in FIG. 1A. The cutouts 31A, 31B extend along a direction substantially parallel to the central axis 12. Preferably, the element 11B is deformable upon crimping pressure applied thereto (which is preferably applied to one or more crimping grooves 33 disposed on the exterior surface of the element 11B) such that the cutout 31B collapses and grasps suture material thread disposed therein as described below.

The suture material can be made of non-stretchable or stretchable, non-absorbable or absorbable materials. The suture material may also be coated with an antibiotic or other therapeutic drug. The suture material can have various outer-diameter or cross-sectional sizes in accordance with the surgical application.

Preferably, the elements 11A, 11B have an annular shape with a diameter on the order of 0.125 inches and a height on the order of 0.1 inches. Moreover, the elements 11A, 11B and the rotating member 13 (and possibly the retention button described below) may be made of non-bioabsorbable material or bioabsorbable material (such as polymers or copolymers of lactide, glycolide, caprolactone, polydioxanone, trimethylene carbonate, polyorthoesters and polyethylene oxide). In addition, the elements 11A, 11B and the rotating member 13 (and possibly the retention button described below) may be coated with an antibiotic or other therapeutic drug. In this configuration, the suturing device 10 of FIGS. 1A and 1B can be used for suturing internal tissues and for microsurgical applications.

During surgical operations, the elements 11A, 11B are initially positioned with respect to one another such that the cutout 31A is substantially aligned with the cutout 31B as shown. At least one suture material length is sewn into tissue in the vicinity of the surgical site. With the two elements 11A, 11B positioned at or near the sewn tissue, one or more segments of the suture material are positioned within the two cutouts 31A and 31B and subject to the desired amount of tension.

While maintaining the desired amount of tension on the suture material, crimping pressure is applied to the element 11B (preferably by applying pressure to the one or more crimping grooves 33) such that the element 11B deforms and the cutout 31B collapses and grasps the suture material thread disposed therein, thereby affixing the element 11B to suture material thread at a position at (or near) the sewn tissue.

The operator inserts the mandrel 21 through the port 19 such that the surfaces of the drive tip 23 mate to the surfaces of the recess 17. The mandrel 21 is rotated such that top element 11A rotates with respect to the bottom element 11B. Such rotation causes the suture material passing through the cutout 31A (and to the collapsed cutout 31B) to wrap around the rotating member 13 in the annular space between the two elements 11A, 11B. The wrapping of the suture material around the rotating member 13, which is preferably formed by one or more complete rotations of the top element 11A with respect to the bottom element 11B, effectively binds the suture material thereto. The one-way rotatability of the two elements 11A, 11B ensures that the suture material is held by the two elements with the desired amount of tension. In this manner, the suturing device of FIGS. 1A and 1B effectively grasps the suture material thread near tissue at a surgical site, and maintains the desired tension on the suture material thread.

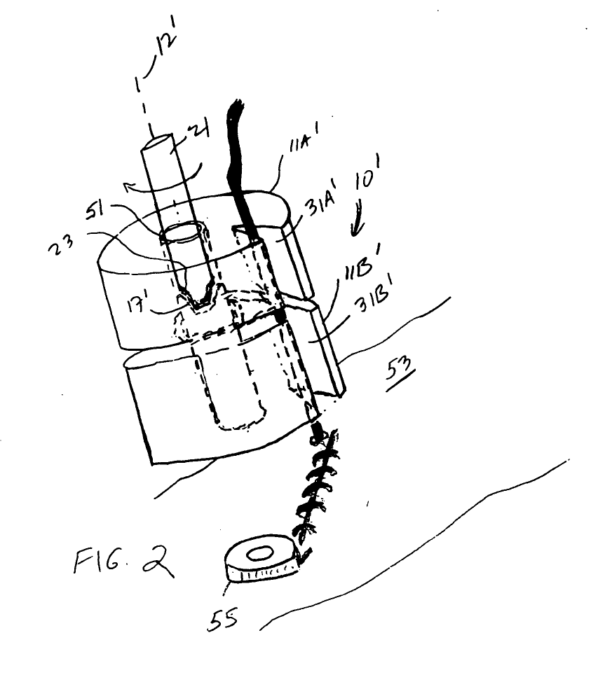

As described below with respect to FIG. 2, one end of the suture material may have a retention button permanently affixed thereto. The shape of the retention button can vary provided that it prevents the suture material from being pulled through the sewn tissue when tension is applied to the opposite end of the suture material. In this configuration, the suture material is sewn through the tissue at the surgical site with tension such that the retention button is disposed adjacent the sewn tissue. The other end of the suture material is then grasped and clamped with tension with the suturing device of FIGS. 1A and 1B to effectively maintain the desired tension on the suture material thread. Alternatively, the retention button may be omitted and replaced by a suture knot or other suitable suture retention mechanism.

An alternate suturing device in accordance the present invention is shown in FIG. 2. The suturing device 10′ includes two elements 11A′, 11B′, preferably of annular shape as shown, that are rotatable with respect to one another about a central axis 12′. Such rotation is preferably accomplished by a central cylinder 51 that extends through a central annular opening in the top element 11A′ and into a central annular opening in the bottom element 11B′. The central annular opening in the bottom element 11B′ is sized to enable the bottom element to rotate about the central cylinder 51, while the central annular opening in the top element 11A′ is sized such that the central cylinder 51 fits snuggly therein. The bottom element 11B′ is held in place along the central axis 12′ by a snap ring or other suitable retention mechanism. The inside of the central cylinder 51 includes a recess portion 17′ that mates to the drive tip 23 of the mandrel 21 for rotating the central cylinder 51 (and the top element 11A′ affixed thereto) with respect to the bottom element 11B′, thereby rotating the top element 11A′ with respect to the bottom element 11B′. Preferably, rotational movement between the two elements 11A′ and 11B′ is limited to one direction (e.g., either counter-clockwise or clockwise) by an annular ridged surface (not shown) that is disposed on the bottom side of the element 11A′ and cooperating pawls (not shown) that are disposed on the top side of the element 11B′ in a manner similar that described above. The ridged surface and the pawls cooperate such that the rotation of the two elements 11A′, 11B′ with respect to one another is limited to one direction (e.g., either counter-clockwise or clockwise).

The two elements 11A′, 11B′ also have cutouts 31A′, 31B′ into their exterior as shown in FIG. 2. The cutouts 31A′, 31B′ extend along a direction substantially parallel to the central axis 12′. Preferably, the element 11B′ is deformable upon crimping pressure applied thereto (which is preferably applied to one or more crimping grooves (not shown) disposed on the exterior surface of the element 11B′) such that the cutout 31B′ collapses and grasps suture material thread disposed therein as described below.

The suture material can be made of non-stretchable or stretchable, non-absorbable or absorbable materials. The suture material may also be coated with an antibiotic or other therapeutic drug. The suture material can have various outer-diameter or cross-sectional sizes in accordance with the surgical application.

Preferably, the elements 11A′, 11B′ have an annular shape with a diameter on the order of 0.125 inches and a height on the order of 0.1 inches. Moreover, the elements 11A′, 11B′ and the cylinder 51 (and possibly the retention button 55 described below) may be made of non-bioabsorbable material or bioabsorbable material (such as polymers or copolymers of lactide, glycolide, caprolactone, polydioxanone, trimethylene carbonate, polyorthoesters and polyethylene oxide). In addition, the elements 11A, 11B and the rotating member 13 (and possibly the retention button 55 described below) may be coated with an antibiotic or other therapeutic drug. In this configuration, the suturing device 10′ of FIG. 2 can be used for suturing internal tissues and for microsurgical applications.

During surgical operations, at least one suture material length is sewn into tissue 53 in the vicinity of the surgical site. Preferably, one end of the suture material length(s) has a retention button 55 permanently affixed thereto. The shape of the retention button 55 can vary provided that it prevents the suture material from being pulled through the sewn tissue 53 when tension is applied to the opposite end of the suture material length. In this configuration, the suture material length is sewn through the tissue at the surgical site with tension such that the retention button is disposed adjacent the sewn tissue 53 as shown. Alternatively, the retention button 55 may be omitted and replaced by a suture knot or other suitable suture retention mechanism.

The two elements 11A′, 11B′ are initially positioned with respect to one another such that the cutout 31A′ is substantially aligned with the cutout 31B′ as shown. With the two elements 11A′, 11B′ positioned at (or near) the sewn tissue 53, one or more segments of the suture material (for example, one shown in FIG. 2) are positioned within the two cutouts 31A′ and 31B′ and subject to the desired amount of tension.

While maintaining the desired amount of tension on the suture material, crimping pressure is applied to the element 11B′ (preferably by applying pressure to the one or more crimping grooves as described above) such that the element 11B′ deforms and the cutout 31B′ collapses and grasps the suture material thread disposed therein, thereby affixing the element 11B′ to the suture material thread at a position near the sewn tissue 53.

The operator then inserts the mandrel 21 into the central cylinder 51 such that the surfaces of the drive tip 23 mate to the surfaces of the recess 17′. The mandrel 21 is rotated such that top element 11A′ rotates with respect to the bottom element 11B′. Such rotation causes the suture material passing through the cutout 31A′ (and to the collapsed cutout 31B′) to wrap around the central cylinder 51 in the annular space between the two elements 11A′, 11B′. The wrapping of the suture material around the central cylinder 51, which is preferably formed by one or more complete rotations of the top element 11A′ with respect to the bottom element 11B′, effectively binds the suture material thereto. The one-way rotatability of the two elements 11A′, 11B′ ensures that the suture material is held by the two elements with the desired amount of tension. In this manner, the suturing device of FIG. 2 effectively grasps the suture material thread near tissue at a surgical site, and maintains the desired tension on the suture material thread.

There have been described and illustrated herein several embodiments of an improved suturing device and a suturing methodology utilizing such devices. While particular embodiments of the invention have been described, it is not intended that the invention be limited thereto, as it is intended that the invention be as broad in scope as the art will allow and that the specification be read likewise. Thus, while particular configurations for guiding and grasping suture material thread and for effectuating rotation of a two suture guiding mechanisms have been disclosed, it will be appreciated that other configurations can be used as well. For example, the top element of the suturing device may also be deformable upon pressure applied thereto such that its cutout collapses and grasps suture material thread disposed therein and the two elements are fixed in position, thereby minimizing the risk of slippage of the suture material thread held therein. It will therefore be appreciated by those skilled in the art that yet other modifications could be made to the provided invention without deviating from its spirit and scope as claimed.

1. A suturing device for surgical applications comprising:
first and second elements that are rotatable with respect to one another about a central axis, each defining a cutout in its exterior surface, the cutouts extending along a direction substantially parallel to the central axis and sized to accept suture thread material therein; and
a central member disposed along the central axis between said first and second elements to define space therebetween;
wherein when said first element is rotated with respect to said second element, the suture thread material wraps around said central member in said space between said first and second elements to thereby grasp and hold the suture thread material therein.

2. A suturing device according to claim 1, wherein:
said second element comprises deformable material such that said cutout of said second element collapses and grasps the suture material thread disposed therein.

3. A suturing device according to claim 2, wherein:
said second element further defines at least one crimping groove for applying pressure to deform said second element.

4. A suturing device according to claim 1, wherein:
said first and second elements have an annular shape.

5. A suturing device according to claim 1, wherein:
said central member comprises a rotating member affixed to said second member that rotates freely about the central axis with respect to said second member.

6. A suturing device according to claim 5, wherein:
said rotating member fits snuggly into a cavity formed in said first element.

7. A suturing device according to claim 6, wherein:
said rotating member includes a recess that is accessible through a port extending along the central axis of said first element.

8. A suturing device according to claim 1, wherein:
said central member comprises a cylinder affixed to said second member that rotates freely about the central axis with respect to said second member.

9. A suturing device according to claim 8, wherein:
said cylinder fits snuggly into an annular recess in said first element.

10. A suturing device according to claim 9, wherein:
said cylinder includes an internal recess that mates to a drive tip of a mandrel inserted therein.

11. A suturing device according to claim 10, wherein:
rotation of the mandrel effectuates rotation of said cylinder and said first element mated thereto.

12. A suturing device according to claim 1, wherein:
rotation of said first element with respect to said second element is limited to one of a counter-clockwise direction and a clockwise direction.

13. A suturing device according to claim 12, wherein:
one of said first element and said second element includes an annular ridged surface and another of said first element and said second element includes at least one pawl disposed opposite said annular ridge surface.

14. A suturing device according to claim 1, wherein:
said first and second elements and said central member are formed from bioabsorbable material.

15. A suturing device according to claim 1, wherein:
said first and second elements have a diameter on the order of 0.125 inches and a height on the order of 0.1 inches.

16. A suturing device according to claim 1, further comprising:
a retention button permanently affixed to the suture material thread.

17. A suturing system comprising:
a suturing device including first and second elements that are rotatable with respect to one another about a central axis, each defining a cutout in its exterior surface, the cutouts extending along a direction substantially parallel to the central axis and sized to accept suture thread material therein, and a central member disposed along the central axis between said first and second elements to define space therebetween, wherein when said first element is rotated with respect to said second element, the suture thread material wraps around said central member in said space between said first and second elements to thereby grasp and hold the suture thread material therein; and
a mandrel that is operably coupled to said first element whereby rotation of said mandrel effectuates rotation said first element.

18. A suturing system according to claim 17, wherein:
said first and second elements have an annular shape.

19. A suturing system according to claim 17, wherein:
said central member comprises a rotating member affixed to said second member that rotates freely about the central axis with respect to said second member.

20. A suturing system according to claim 19, wherein:
said rotating member fits snuggly into a cavity formed in said first element.

21. A suturing system according to claim 18, wherein:
said rotating member includes a recess that is accessible through a port extending along the central axis of said first element; and
said mandrel is inserted through said port and has a drive tip that mates to said recess, wherein rotation of the mandrel effectuates rotation of the rotating member and said first element mated thereto.

22. A suturing system according to claim 17, wherein:
said central member comprises a cylinder affixed to said second member that rotates freely about the central axis with respect to said second member.

23. A suturing system according to claim 22, wherein:
said cylinder fits snuggly into an annular recess in said first element.

24. A suturing system according to claim 22, wherein:
said cylinder includes an internal recess; and
said mandrel is inserted into said cylinder and has a drive tip that mates to said internal recess, wherein rotation of the mandrel effectuates rotation of said cylinder and said first element mated thereto.

25. A suturing system according to claim 17, further comprising:
a retention button permanently affixed to the suture material thread.

26. A method of suturing tissue comprising:
sewing suture material thread through the tissue;
providing a suturing device comprising first and second elements that are rotatable with respect to one another about a central axis, each defining a cutout in its exterior surface, the cutout extending along a direction substantially parallel to the central axis and sized to accept said suture material thread therein, and a central member disposed along the central axis between said first and second elements to define space therebetween, wherein said first and second elements are initially positioned with respect to one another such that said cutouts are substantially aligned with one another;
positioning at least one segment of the suture material thread within said cutouts and subjecting said at least one segment of the suture material thread to a desired amount of tension; and
rotating said first element with respect to said second element to wrap suture material thread around said central member in said space between said first and second elements to thereby grasp and hold suture material thread therein.

27. A method according to claim 26, further comprising the step of:
prior to rotating said first element, deforming said second element such that the cutout of said second element collapses and grasps suture material thread at a point substantially adjacent sewn tissue.

28. A method according to claim 26, wherein:
rotation of the first element is effectuated by rotation of a mandrel with a drive tip that mates to a recessed element of said suturing device.

29. A method according to claim 26, wherein:
rotation of said first element with respect to said second element is limited to one of a counter-clockwise direction and a clockwise direction.

30. A method according to claim 26, wherein:
said first and second elements and said central member are formed from bioabsorbable material.

31. A method according to claim 26, wherein:
said first and second elements have a diameter on the order of 0.125 inches and a height on the order of 0.1 inches.

32. A method according to claim 26, further comprising:
providing suture material thread with a retention button permanently affixed to an end of the suture material thread; and
sewing the surgical material thread in the tissue such that said retention button is disposed adjacent the sewn tissue and the suturing device grasps the other end of the suture material thread.
